(19) United States
(12) Patent Application Publication
(10) Pub. No.: US 2026/0224143 A1
(43) Pub. Date: Aug. 6, 2026

(54) WEARABLE MUSCLE STRENGTH MEASURING DEVICE AND GRAVITY COMPENSATION MECHANISM-BASED MUSCLE STRENGTH MEASURING METHOD USING SAME

(71) Applicant: SEOUL NATIONAL UNIVERSITY HOSPITAL, Seoul (KR)

(72) Inventors: Sungwan KIM, Yongin-si (KR); Minwoo CHO, Seoul (KR); Youho MYONG, Seoul (KR); Sungwoo PARK, Seoul (KR); Byung-Mo OH, Goyang-si (KR); Woohyung LEE, Seoul (KR); Seungyeon CHO, Incheon (KR)

(73) Assignee: SEOUL NATIONAL UNIVERSITY HOSPITAL, Seoul (KR)

(21) Appl. No.: 19/123,611

(22) PCT Filed: Aug. 24, 2023
(86) PCT No.: PCT/KR2023/012569

§ 371 Date: Apr. 23, 2025

(30) Foreign Application Priority Data
Sep. 2, 2022 (KR) 10-2022-0111266

(51) Int. Cl. A61B 5/22; A61B 5/00; G01G 19/44

(52) U.S. Cl. CPC A61B 5/224; A61B 5/4528; A61B 5/6831; A61B 5/6843; A61B 5/742; G01G 19/44; A61B 2560/0223; A61B 2562/0252; A61B 2562/0261

(57) ABSTRACT

measuring device. The wearable muscle strength measuring device includes: a rotation shaft configured to be located on a joint part of a subject; a first lever arm extending in a first direction; a second lever arm extending in a second direction; a first contact part fixed to the first lever arm and formed into a curved surface so that the proximal portion of the upper or lower limb of a subject is seated thereon; a second contact part fixed to the second lever arm and formed into a curved surface so that the distal portion of the upper or lower limb of the subject is seated thereon; and a sensor unit configured to measure at least one of the muscle strength of the upper or lower limb and the weight of the upper or lower limb.

### TECHNICAL FIELD

The present disclosure relates to a wearable muscle strength measuring device and a gravity compensation mechanism-based muscle strength measuring method using the same.

The national research and development projects that supported this disclosure are as follows.

The project management agency and project implementation agency are Seoul National University Hospital, the project name is ‘Super-generation Research Lab Project’, the project name is ‘Establishment of a Convergence Electronics Lab for the Development of Next-Generation Medical Devices’, and the project period is 2023 Jun. 1~2026 May 31. This study was supported by grant no. 1920230020 from the SNUH Research Fund.

The project management agency and project implementation agency are Seoul National University Hospital, the project name is ‘General Research Project’, the project name is ‘Development and Clinical Verification of Portable Dynamometer’, and the project period is 2021 Aug. 01~2022 Jul. 31. This study was supported by grant no. 0420212130 from the SNUH Research Fund.

The project management agency is the National Research Foundation of Korea, the project implementing agency is Seoul National University Hospital, the project name is ‘Individual Basic Research (Ministry of Science and ICT) ’, the project name is ‘GMP Approval through Development of Portable Dynamometer and Clinical Verification’, and the project period is 2022 Mar. 01~2023 Feb. 28. The project unique number is 1711162130, and the project number is 2021R1C1C2095529.

### BACKGROUND ART

Muscle strength is used as an important indicator for patient walking, fall prediction, and the determination of functional prognosis. More specifically, muscle strength is necessary to quantify the results of rehabilitation treatment for acute neurological hospitalization patients, and is also necessary to measure sarcopenia in the elderly.

As a commonly used muscle strength measuring device, an isokinetic dynamometer from Biodex Company is a medical device that measures and evaluates the muscle strength of a patient's body, the range of motion of a joint, etc. by using driving force. A subject performs tests and exercises and then records the measurements obtained from the equipment by using the isokinetic dynamometer so that medical staff evaluates them. It consists of a dynamometer, a chair whose position is adjustable, a controller, dynamometer accessories, and internal software for quantitative measurement. The device can measure the speed of movement and torque, and equipment for measuring the upper limb and equipment for measuring the lower limbs are spread apart from each other. The equipment is bulky and fixedly installed, so that it is difficult to move the equipment, making it inconvenient to use in hospital beds or outpatient clinics. Furthermore, subjects need to move to examination rooms on their own, so that there is a problem in that it is difficult to apply the equipment for hospitalized patients who have difficulty moving.

Accordingly, in actual medical sites, there is used a manual muscle test (MMT) in which a measurer holds a subject's body with his or her hand and qualitatively records the force of flexion or extension. However, this method has a disadvantage in that the measured value may vary depending on the level of skill of the medical staff. Furthermore, the upper and lower limbs need to be measured with different pieces of equipment, so that the initial purchase, maintenance, and repair costs are high. Accordingly, although this equipment is the only equipment that can quantitatively evaluate a patient's muscle joint movement function, it is not widely utilized.

Therefore, there is a demand for a muscle strength measuring device that is easy to carry, can be used conveniently in clinical practice, and can quantify and measure muscle strength.

### DISCLOSURE

### Technical Problem

The present disclosure has been contrived in response to the above-described background technology, and is directed to a wearable muscle strength measuring device that may be worn on the upper r lower limb and a gravity compensation mechanism-based muscle strength measuring method using the same.

However, the objects to be accomplished by the present disclosure are not limited to the object mentioned above, and other objects not mentioned may be clearly understood based on the following description.

### Technical Solution

Disclosed herein is a wearable muscle strength measuring device according to one embodiment of the present disclosure for achieving the above-described object. The wearable muscle strength measuring device includes: a rotation shaft configured to be located on a joint part of a subject; a first lever arm extending in a first direction based on the rotation shaft; a second lever arm extending in a second direction different from the first direction based on the rotation shaft; a first contact part fixed to the first lever arm and formed into a curved surface so that the proximal portion of the upper or lower limb of a subject is seated thereon; a second contact part fixed to the second lever arm and formed into a curved surface so that the distal portion of the upper or lower limb of the subject is seated thereon, and having an area smaller than or equal to that of the first contact part; and a sensor unit coupled to the rotation shaft or the second lever arm, and configured to measure at least one of the muscle strength of the upper or lower limb and the weight of the upper or lower limb.

Alternatively, the wearable muscle strength measuring device further includes: a control unit configured to perform a gravity compensation mechanism by adjusting the zero point of the sensor unit based on the weight of the upper or lower limb measured in the state where the muscle strength of the subject is not exerted and calculate the muscle strength of the upper or lower limb.

Alternatively, the wearable muscle strength measuring device further includes a motor unit configured to spread apart the first and second lever arms while rotating them at a preset angular velocity around the rotation shaft, and the sensor unit measures the muscle strength of the upper or lower limb that resists the movement of the first and second lever arms.

Alternatively, the sensor unit measures the muscle strength of the upper or lower limb at a preset angle corresponding to the type of joint of the subject.

Alternatively, the wearable muscle strength measuring device further includes: a ratchet gear having the rotation shaft as its central axis; and a stopper configured to perform hooking engagement with the ratchet gear at the preset angle.

Alternatively, the sensor unit includes a load cell including a strain gauge, the fixation portion of the load cell is located on the rotation shaft, and the force application point of the load cell is located on the bottom surface of the second contact part.

Alternatively, the first and second contact parts each further include: a cylindrical frame configured such that the upper or lower limb of the subject is inserted thereinto; and a close contact part provided inside the frame so that the upper or lower limb of the subject is maintained in contact while the upper or lower limb of the subject moves.

Alternatively, the wearable muscle strength measuring device further includes: a plurality of close contact sensors spaced apart at preset intervals inside the close contact part to detect whether the upper or lower limb is maintained in contact.

Alternatively, the close contact part includes a cushion including non-Newtonian liquid or an air pressure pump, and the cushion is deformed in accordance with a thickness of the upper or lower limb of the subject.

Alternatively, the wearable muscle strength measuring device further includes a support configured to fix the wearable muscle strength measuring device so that the direction of movement of the joint of the subject is perpendicular to the direction of gravity, and the control unit modifies the gravity compensation mechanism according to the location at which the support is installed and the form in which the wearable muscle strength measuring device is coupled with the support.

Alternatively, the wearable muscle strength measuring device further includes: a display unit configured to display the magnitude of at least one of the measured value, measured by the sensor unit, and the muscle strength of the upper or lower limb, calculated by the control unit, according to the movement of the subject, as a color code and distinctively display the point at which the magnitude is maximum.

Alternatively, each of the first and second contact parts includes: a plurality of support plates each formed as a curved surface, and each provided with slots through which straps for fixing the upper or lower limb pass; and length adjustment parts configured to connect the individual support plates.

Alternatively, the first and second lever arms are folded to face the same direction based on the rotation shaft.

Disclosed herein is a muscle strength measuring method, the muscle strength measuring method being performed by a wearable muscle strength measuring device, according to one embodiment of the present disclosure for achieving the above-described object. The muscle strength measuring method includes: measuring the weight of the upper or lower limb of a subject in the state where the subject's muscle strength is not exerted; performing a gravity compensation mechanism on the wearable muscle strength measuring device by adjusting the zero point of a sensor unit based on the measured weight; and measuring the muscle strength of the upper or lower limb that resists the movement of lever arms rotating around the rotation shaft of the wearable muscle strength measuring device, or measuring the muscle strength of the upper or lower limb at a preset angle corresponding to the type of joint of the subject.

Alternatively, measuring the weight of the upper or lower limb includes: checking whether the direction of movement of the joint of the subject and the direction of gravity are perpendicular to each other; and, when they are perpendicular, determining a value measured by the sensor unit to be the weight of the upper or lower limb, and, when they are not perpendicular, measuring the angle formed by the direction of movement of the joint of the subject and the direction of gravity, and calculating the weight of the upper or lower limb based on the angle and the value measured by the sensor unit.

### Advantageous Effects

According to the present disclosure, the muscle strength of the upper or lower limb may be measured with a single device, and the size and length of the device may be easily adjusted to suit the physical characteristics of the subject, thereby increasing the convenience of use.

In addition, the wearable muscle strength measuring device according to the present disclosure is lightweight and portable, which increases convenience in hospital beds and outpatient clinics where muscle strength needs to be measured, so that the inconvenience in which patients, who are subjects, have to move to separate examination rooms is reduced. Furthermore, as the time and manpower required for examination are reduced, response to patients in emergency situations may be performed effectively.

### DESCRIPTION OF DRAWINGS

FIG. 1 is a block diagram showing a wearable muscle strength measuring device according to one embodiment of the present disclosure;

FIG. 2 is a perspective view showing the wearable muscle strength measuring device according to the one embodiment of the present disclosure;

FIG. 3 is an exemplary diagram showing a form in which the wearable muscle strength measuring device according to the one embodiment of the present disclosure is worn;

FIG. 4 is an exemplary diagram showing a form in which the wearable muscle strength measuring device according to the one embodiment of the present disclosure is folded;

FIGS. 5 and 6 are exemplary diagrams showing the close contact part of a wearable muscle strength measuring device according to one embodiment of the present disclosure;

FIG. 7 is a perspective view showing a wearable muscle strength measuring device according to one embodiment of the present disclosure;

FIGS. 8 and 9 are perspective views illustrating a wearable muscle strength measuring device according to one embodiment of the present disclosure;

FIG. 10 a flowchart showing a muscle strength measuring method of a wearable muscle strength measuring device according to one embodiment of the present disclosure; and

FIG. 11 is an exemplary diagram showing the forces acting on a wearable muscle strength measuring device according to one embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “at least one of A and B” used herein should be interpreted to refer to all of A, B, and combinations of A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.” The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “connected” used herein should be interpreted to include not only a case where components are “directly connected” but also a case where another component is “present” between components and a case where components are “electrically connected” to each other with another component interposed therebetween.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

In the present specification, a wearable muscle strength measuring device refers to a device that is worn on the upper or lower limb of a subject and measures the muscle strength of a main joint or a knee joint. The wearable muscle strength measuring device may be worn on a portion of a body part of a subject by a measurer, or may be worn by a subject himself or herself.

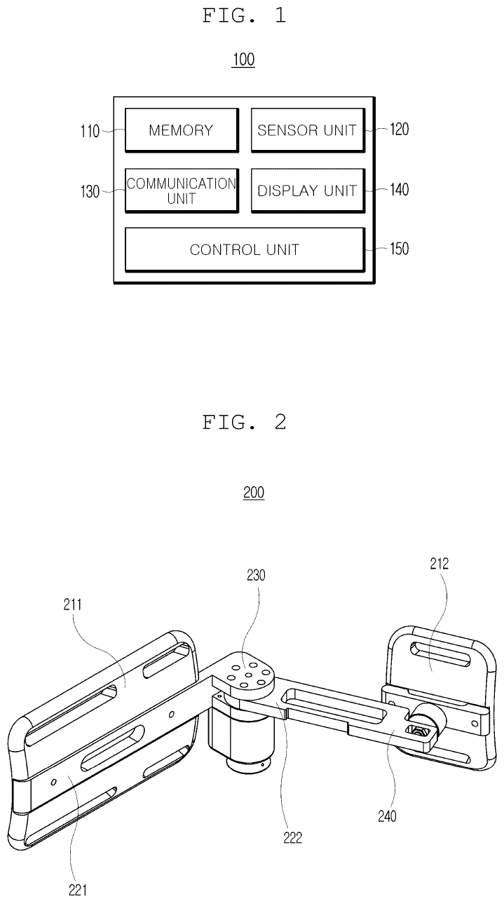

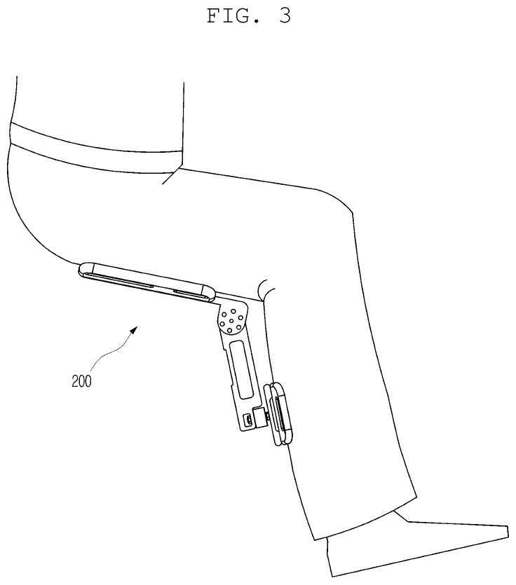

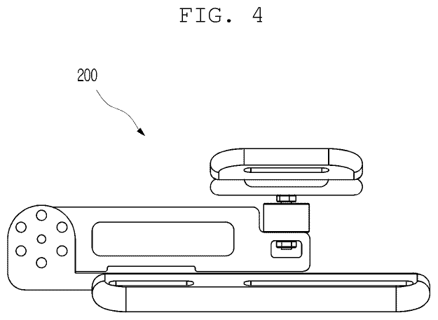

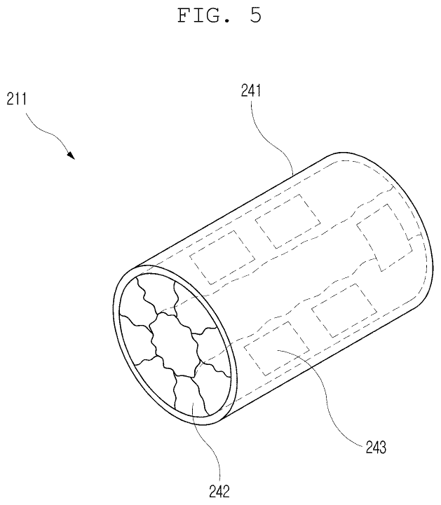

FIG. 1 is a block diagram showing a wearable muscle strength measuring device according to one embodiment of the present disclosure, FIG. 2 is a perspective view showing the wearable muscle strength measuring device according to the one embodiment of the present disclosure, FIG. 3 is an exemplary diagram showing a form in which the wearable muscle strength measuring device according to the one embodiment of the present disclosure is worn, and FIG. 4 is an exemplary diagram showing a form in which the wearable muscle strength measuring device according to the one embodiment of the present disclosure is folded.

Referring to FIG. 1, a wearable muscle strength measuring device 100 may include memory 110, a sensor unit 120, a communication unit 130, a display unit 140, and a control unit 150.

The memory 110 stores a program in which the operation of the wearable muscle strength measuring device 100 is recorded. The memory 110 stores a sensing value that is measured by the sensor unit 120, and the gravity compensation mechanism that is applied to the sensing value and calculates muscle strength. The memory 110 stores the information of a subject and the values measured by the sensor unit 120. For example, the information of a joint on which the wearable muscle strength measuring device 100 is worn, a body part where muscle strength is measured, and the gender, age, age group, previous hospital records, past muscle strength, and/or the like of a subject are stored for each subject. This is collectively referred to as measured data.

The measured data may be stored in an external server via the communication unit 130. The external server may be, e.g., a server within a hospital, and may be included in an electronic medical record (EMR). The measured data may be managed together with the data generated in a hospital, such as a subject's body temperature, blood pressure, and oxygen saturation, and may be provided to clinical medical staff and nursing staff.

The external server may be, e.g., an application server that provides measured data to the subject's terminal. The application server may check the measured data, provided by the subject's application server, on the terminal. Accordingly, the trend of measured data may be checked, and the management of muscle strength may be actively performed.

The subject's terminal may be a wireless communication device ensuring portability and mobility, and may be any type of handheld-based wireless communication device such as a smartphone, a tablet PC, or a laptop. Furthermore, the ‘terminal’ may also be a wired communication device, such as a PC, capable of connecting to another terminal or a server over a network.

The memory 110 may perform a function of temporarily or permanently storing the data processed and generated in the wearable muscle strength measuring device 100. The memory 110 is a general term for a nonvolatile storage device that maintains stored information even when power is not supplied and a volatile storage device that requires power to maintain stored information, but the scope of the present invention is not limited thereto.

The sensor unit 120 may measure the muscle strength of the subject in various modes. The sensor unit 120 may measure the force applied to the wearable muscle strength measuring device 100 by the upper or lower limb of the subject, or may measure the torque rotated by the upper or lower limb.

The sensor unit 120 may include at least one of various types of sensors, such as a load cell, a torque sensor, and a strain gauge.

The load cell measures the elasticity changing when force is applied and outputs the value as an electrical signal. The load cell may include a strain gauge made of an aluminum alloy such as stainless steel. The load cell measures the force with which a first lever arm 221 and a second lever arm 222 bend the load cell. The fixation portion of the load cell is located on a rotation shaft 230, and the force application point of the load cell may be located on the bottom surface of a second contact part 212.

The torque sensor is provided on the rotation shaft 230, and measures the torque at which the first and second lever arms 221 and 222 attempt to rotate. The control unit 150 calculates the muscle strength by dividing a measured torque value by the distance between the torque sensor and the application point at which the upper or lower limb makes contact on the first and second lever arms 221 and 222.

The strain gauge is a sensor that outputs a current that changes when a tensile force is applied to a gauge and the gauge expands or contracts. The control unit 150 calculates muscle strength based on the elastic moduli of the first and second lever arms 221 and 222 and the change in the amount of current of the strain gauge.

The sensor unit 120 may operate according to the set muscle strength measurement mode. For example, an isokinetic muscle strength measurement mode is a mode in which measurement is started at the physiological rest angle of a joint, rotation is performed at a constant angular velocity, and the force resisting a torque sensor is measured. For example, an isometric muscle strength measurement mode is a mode in which a torque sensor is fixed at a preset angle and the torque applied by a subject is measured to thus calculate the muscle strength. In this case, the design is made such that the maximum muscle strength is measured at the angle at which the joints of the human body can exhibit maximum contraction force, for example, 150 degrees in the case of the extension of the elbow joint and 100 degrees in the case of the extension of the knee joint, which are recorded in existing literature.

The wearable muscle strength measuring device 100 may further include a motor unit. The motor unit spreads apart the first and second lever arms 221 and 222 while rotating them at a preset angular velocity according to the muscle strength measurement mode. The motor unit may operate in an isokinetic muscle strength measurement mode.

The communication unit 130 transmits and receives measured data to and from an external computing device over a network. The network refers to a connection structure that enables information exchange between individual nodes such as terminals and servers, and includes a local area network (LAN), a wide area network (WAN), the Internet (WWW), a wired and wireless data communication network, a telephone network, a wired and wireless television communication network, etc. Examples of the wireless data communication network include, but are not limited to, 3G, 4G, 5G, 3rd Generation Partnership Project (3GPP), Long Term Evolution (LTE), Worldwide Interoperability for Microwave Access (WIMAX), Wi-Fi, Bluetooth communication, infrared communication, ultrasonic communication, visible light communication (VLC), and Li-Fi networks, etc.

The display unit 140 displays the magnitude of at least one of the measured value, measured by the sensor unit 120, and the muscle strength of the upper or lower limb, calculated by the control unit 150, according to the movement of the subject, as a color code, and distinctively displays the point where the magnitude is maximum. For example, the display unit 140 displays an image where a bar is filled when the muscle strength increases, and an image where a bar is emptied when the muscle strength decreases. When the muscle strength is equal to the gravity applied to the upper or lower limb, a zero point is displayed. When the muscle strength is lower than the gravity applied to the upper or lower limb, a green color is displayed. In contrast, when the muscle strength is higher than the gravity, a red color is displayed. Meanwhile, the display method is not limited thereto. For example, the changes in muscle strength may be displayed in a graph, e. g., 2 histogram. Alternatively, the changing muscle strength may be displayed numerically. The muscle strength changing in real time may be displayed, or the muscle strength at a preset time or at a preset angle may be displayed.

The control unit 150 controls the overall operation of the wearable muscle strength measuring device 100. The control unit 150 controls the operation of the sensor unit 120, and calculates the muscle strength of the subject based on the value measured by the sensor unit 120. The control unit 150 may adjust the zero point of the sensor unit 120 or modify the gravity compensation mechanism using the hospital records of the subject, who is the main user of the wearable muscle strength measuring device 100.

The control unit 150 controls the operation of at least one of the sensors included in the sensor unit 120 according to the muscle strength measurement mode. The motor unit may be additionally controlled such that the sensor unit 120 operates. For example, when the muscle strength measurement mode is set to the isokinetic muscle strength measurement mode, the torque sensor is operated and the first and second lever arms 221 and 222 are controlled to rotate at a constant speed. In this case, the rotation speed is set based on the information of the subject or the measurement data stored in advance. When the muscle strength measurement mode is set to the isometric muscle strength measurement mode, the torque sensor is fixed at a preset angle based on the information of the subject or the measured data.

The control unit 150 operates a plurality of sensors, and may calculate muscle strength by combining sensing values obtained from the individual sensors. In this case, different weights may be allocated to individual sensing values. For example, the weights vary depending on the muscle strength measurement mode, the muscle strength measurement site, the type of joint, and the measurement target data.

The control unit 150 measures the weight of the subject's upper or lower limb, or adjusts the zero point of the sensor unit 120 by using the weights stored in advance. The control unit 150 adjusts the zero point based on the subject's medical data, such as the age, gender, age group, and past medical history of the subject, and the weight of the wearable muscle strength measuring device 100.

The control unit 150 performs the overall gravity compensation mechanism Of the wearable muscle strength measuring device 100.

A subject having a lower upper or lower limb weight may easily overcome the gravity applied to the upper or lower limb, so that the muscle strength is measured as a larger value than an actual value. In contrast, for a subject having a higher upper or lower limb weight, the muscle strength is measured as a smaller value, making it difficult to predict the prognosis. Accordingly, the gravity compensation mechanism refers to a method for physically preventing a subject's movement from being affected by gravity during a muscle strength measuring process or a method for calculating the muscle strength by reflecting the weight of a subject's upper or lower limb in a measured sensing value.

More specifically, the control unit 150 measures the weight of the upper or lower limb in the state where a subject's muscle strength is not exerted. The zero point of the sensor unit 120 is adjusted based on this value. In order to generate the state where a subject's muscle strength is not exerted, a support 250 may be installed in the wearable muscle strength measuring device 100 so that the direction of movement of the subject's joint is perpendicular to the direction of gravity.

The control unit 150 may include all types of devices capable of processing data. For example, the control unit 150 may refer to a hardware-embedded data processing device having a physically structured circuit to perform the functions described by codes or instructions included in a program. Examples of the hardware-embedded data processing device may include processing devices such as a microprocessor, a central processing unit (CPU), a processor core, a multiprocessor, an application-specific integrated circuit (ASIC), a field programmable gate array (FPGA), etc., but the scope of the present invention is not limited thereto.

Clinically, the muscle strength of the distal or proximal wrist joint, the radiocarpal joint, the elbow joint, the knee joint, the hip joint, or the like is measured. Accordingly, the first and second contact parts 211 and 212 are worn on the distal and proximal flat bones of a joint to be measured, respectively. The sensor unit 120 is seated on the left and right flexion and extension rotation shaft 230 of each joint. Accordingly, the wearable muscle strength measuring device 100 may measure the muscle strength of the shoulder joint and the elbow joint, and the muscle strength of the multiple joints of the elbow joint, the radiocarpal joint, and the carpometacarpal joint.

Referring to FIG. 2, the wearable muscle strength measuring device 200 includes: the rotation shaft 230 configured to be located on a joint part of a subject, the first lever arm 221 extending in a first direction based on the rotation shaft 230, the second lever arm 222 extending in a second direction different from the first direction based on the rotation shaft 230, the first contact part 211 fixed to the first lever arm 221 and formed into a curved surface so that the proximal portion of the upper or lower limb of a subject is seated thereon, the second contact part 212 fixed to the second lever arm 222 and formed into a curved surface so that the distal portion of the upper or lower limb of the subject is seated thereon, and having an area smaller than or equal to that of the first contact part 211, and the sensor unit 240 coupled to the rotation shaft 230 or the second lever arm 222 and configured to measure at least one of the muscle strength of the upper or lower limb and the weight of the upper or lower limb.

The driving angle of the first and second lever arms 221 and 222 is set by the control unit 150. The driving angle is determined depending on whether a measurement target site is the upper or lower limb. The control unit 150 sets the driving angle based on the physiological rest angle of each joint. For the safety of a subject, the first and second lever arms 221 and 222 operate within a range that does not exceed the set angle.

Referring to FIG. 3, when the lower limb is measured, the top surface of the first contact part 211 and the top surface of the second contact part 212 form an angle exceeding 180 degrees with respect to the rotation shaft 230. The proximal and distal portions of the lower limb are seated on the top surface of the first contact part 211 and the top surface of the second contact part 212, respectively. While the muscle strength is measured, the angle between the top surface of the first contact part 211 and the top surface of the second contact part 212 approaches 360 degrees.

Although not shown, when the upper limb is measured, the top surface of the first contact part 211 and the top surface of the second contact part 212 may form an angle not exceeding 180 degrees with respect to the rotation shaft 230. The proximal and distal portions of the upper limb are seated on the top surface of the first contact part 211 and the top surface of the second contact part 212, respectively. While the muscle strength is measured, the angle between the top surface of the first contact part 211 and the top surface Of the second contact part 212 approaches 0 degrees.

Meanwhile, the second contact part 212 and the sensor unit 240 may be coupled via a ball joint (not shown). For example, the angle formed by the second contact part 212 and the sensor unit 240 may vary depending on the posture of the subject, and accordingly, the ball joint may be rotated according to the direction. When the subject stretches his/her leg, the sensor unit 240 is subjected to a tensile force. In this case, the ball joint may minimize the moment force by maintaining the direction of the tensile force and the direction of the sensor unit 240. When the subject bends his/her leg, the sensor unit 240 is subjected to a compressive force. In this case, by restricting the degree of freedom to limit the rotation of the ball joint, unwanted movement may be prevented.

The first and second contact parts 211 and 212 may each include one or more slots through which straps for fixing the upper or lower limb pass. The first contact part 211 may have longer slots than the second contact part 212, or may have a larger number of slots than the second contact part 212.

Although the first contact part 211 is shown as a single component in FIG. 1, the first contact part 211 may be provided with a plurality of smaller-sized contact portions.

Since the circumference or thickness of the proximal portion is generally larger than that of the distal portion, the length of the first lever arm 221 may be equal to or longer than that of the second lever arm 222 for the convenience of use and satisfaction of the subject.

The display unit 140 may be provided on one of the rotation shaft 230, the first lever arm 221, and the second lever arm 222.

The motor unit may be provided at a location where it is easy to rotate the first lever arm 221 or the second lever arm 222.

Referring to FIG. 4, the first and second lever arms 221 and 222 are folded to face the same direction based on the rotation shaft 230. That is, the wearable muscle strength measuring device 200 may be folded such that the bottom surface of the first contact part 211 and the bottom surface of the second contact part 212 face each other.

Conventional muscle strength measuring devices are large in size and heavy in weight, so that it is difficult to move them. The wearable muscle strength measuring device 200 according to the present disclosure increases convenience in hospital beds and outpatient clinics equipped with the wearable muscle strength measuring device 200 because the volume thereof is reduced, and thus, the portability thereof is increased. Accordingly, the inconvenience in which patients, who are subjects, have to move to separate examination rooms is reduced. Furthermore, as the time and manpower required for examination are reduced, response to patients in emergency situations may be performed effectively.

Meanwhile, the wearable muscle strength measuring device 200 needs to be fixed at a specific angle with respect to the rotation shaft 230. For example, in the isokinetic muscle strength measurement mode, the physiological rest angle of the joint needs to be set, and, in the isometric muscle strength measurement mode, the torque sensor needs to be fixed at a preset angle.

Accordingly, there may be provided a ratchet gear (not shown) having the rotation shaft 230 as a central axis and a stopper (not shown) capable of fixing the angle of the ratchet gear. For the sake of user convenience, the subject wears the wearable muscle strength measuring device 200 with the ratchet gear and the stopper disengaged from each other, and the subject or a measurer may engage the ratchet gear and the stopper with each other at a preset angle and then start measurement after wearing the device. Due to this structure, the wearable muscle strength measuring device 200 may be worn on joints having various angles.

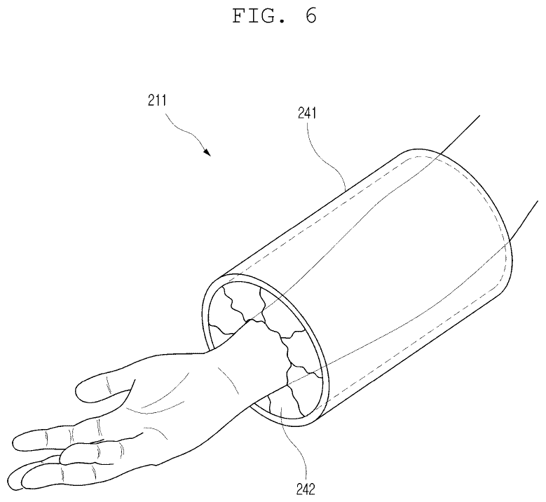

FIGS. 5 and 6 are exemplary diagrams showing the close contact part of a wearable muscle strength measuring device according to one embodiment of the present disclosure.

Referring to FIG. 5, the first and second contact parts 211 and 212 may differ from the shapes of FIGS. 2 to 4. For example, they may each have a cylindrical shape through which the upper or lower limb passes. The first contact part 211 will be described below, but the same applies to the second contact part 212.

The first contact part 211 includes a cylindrical frame 241 configured such that the upper or lower limb of the subject is inserted thereinto, and a close contact part 242 provided inside the frame 241 so that the upper or lower limb of the subject is maintained in contact while the upper or lower limb of the subject moves.

The close contact part 242 is composed of a cushion containing a fluid therein. The fluid may be a non-Newtonian liquid or air. When the fluid is air, the close contact part 242 includes an air pressure pump. The cushion is deformed in accordance with the thickness of the upper or lower limb of the subject.

A plurality of close contact sensors 243 may be further provided inside the close contact unit 242. The individual close contact sensors 243 are spaced apart at preset intervals to detect whether the upper or lower limb is maintained in contact. The control unit 150 receives information about close contact or not from the close contact sensors 243, and informs the subject of contact or not via the display unit 140. The control unit 150 measures the muscle strength and weight of the upper or lower limb by considering contact or not.

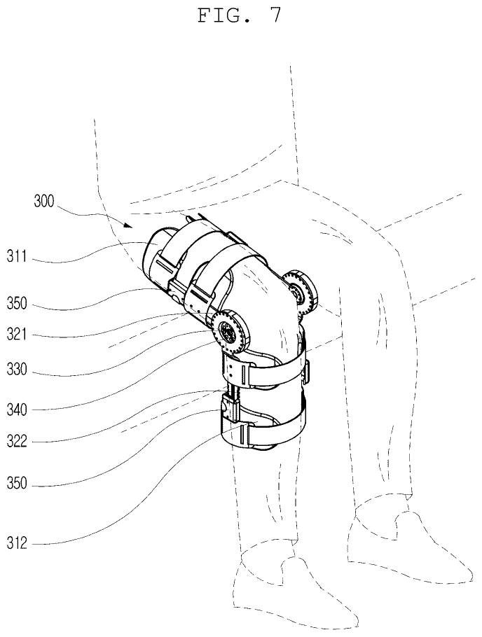

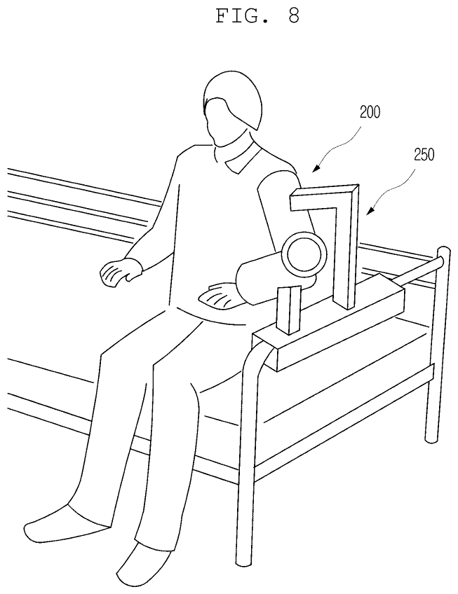

FIG. 7 is a perspective view showing a wearable muscle strength measuring device according to one embodiment of the present disclosure.

Referring to FIG. 7, a wearable muscle strength measuring device 300 is similar to the wearable muscle strength measuring device 200 of FIGS. 2 to 4, so that only the differences therebetween will be described.

The wearable muscle strength measuring device 300 may include two units, and the individual units may be connected to each other by a band or the like and fixed to and worn on the upper or lower limb of the subject. Since the two units have symmetrical structures, only one unit will be described below.

The wearable muscle strength measuring device 300 includes a first lever arm 321 and a second lever arm 322 that extend around a rotation shaft 330, and a first contact part 311 and a second contact part 312 are coupled to the respective lever arms.

The first contact part 311 and the second contact part 312 are formed as curved surfaces, respectively, and each include a plurality of support plates having slots through which straps for fixing the upper or lower limb pass. The individual support plates are made of a flexible material, and the size of the support plates of the first contact part 311 may be equal to or larger than that of the support plates of the second contact part 312.

Parts of the first and second lever arms 321 and 322 each include a length adjustment part 350 that connects individual support plates. Since the length is adjusted according to the age, height, and muscle strength measurement portion of the subject, user convenience is increased. Since the one device may be used in various situations, excessive expenditure may be prevented even in a hospital equipped with the wearable muscle strength measuring device 300.

The sensor unit 340 of the wearable muscle strength measuring device 300 includes a torque sensor. Accordingly, the center of the sensor unit 340 may be the same as the rotation axis 330.

The wearable muscle strength measuring device 300 may be provided with a ratchet gear (not shown) having the rotation shaft 330 as a central axis and a stopper (not shown) capable of fixing the angle of the ratchet gear, like the wearable muscle strength measuring device 200.

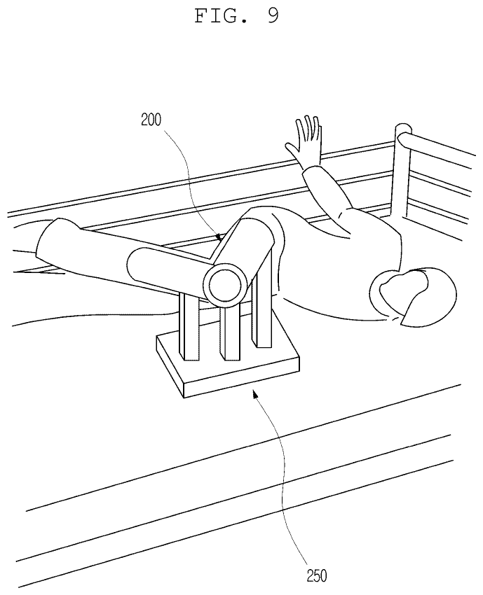

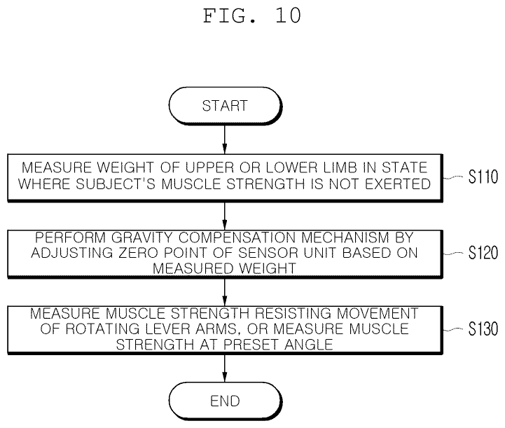

FIGS. 8 and 9 are perspective views illustrating a wearable muscle strength measuring device according to one embodiment of the present disclosure.

The wearable muscle strength measuring device 200 of FIGS. 8 and 9 may be the wearable muscle strength measuring device 200 of FIGS. 2 to 4 or the wearable muscle strength measuring device 300 of FIG. 7. The wearable muscle strength measuring device 200 further includes a support 250 that supports the wearable muscle strength measuring device 200 so that the direction of movement of a subject's joint is perpendicular to the direction of gravity.

Referring to FIG. 8, when the muscle strength of the upper limb is measured, the support 250 is coupled to the wearable muscle strength measuring device 200 so that the elbow joint moves in a direction parallel to a horizontal plane. The support 250 is coupled to a bed frame.

Referring to FIG. 9, when the muscle strength of the lower limb is measured, a support 250 is coupled to a wearable muscle strength measuring device 200 so that the knee joint moves in a direction parallel to a horizontal plane. The support 250 includes a pedestal so that it is stably fixed to a floor forming the horizontal plane. The height and angle of the pedestal are determined according to the physical characteristics of a subject.

The control unit 150 performs a gravity compensation mechanism by considering the weight of the support 250, the form in which the support 250 and the wearable muscle strength measuring device 200 are coupled with each other, and the weight of the wearable muscle strength measuring device 200. For example, the control unit 150 calculates muscle strength by modifying the sensing value based on the weight of the wearable muscle strength measuring device 200 supported by the support 250 and the magnitude and angle of the forces acting between the support 250 and the wearable muscle strength measuring device 200.

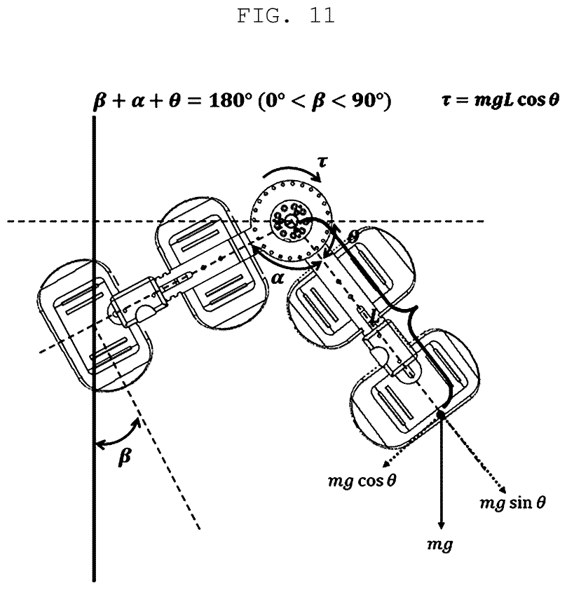

FIG. 10 is a flowchart showing a muscle strength measuring method of a wearable muscle strength measuring device according to one embodiment of the present disclosure, and FIG. 11 is an exemplary diagram showing the forces acting on a wearable muscle strength measuring device according to one embodiment of the present disclosure.

Referring to FIG. 10, the wearable muscle strength measuring device measures the weight of the upper or lower limb of a subject in the state in which the subject's muscle strength is not exerted in step S110.

In this case, the wearable muscle strength measuring device checks whether the direction of movement of the subject's joint and the direction of gravity are vertical to each other. When they are vertical, the sensing value measured by the sensor unit 240 is determined to be the weight of the upper or lower limb. When they are not vertical, the angle formed by the direction of movement of the subject's joint and the direction of gravity is measured, and the weight of the upper or lower limb is calculated based on the measured angle and the sensing value.

Referring to FIG. 11, α is the angle formed by the first and second lever arms 221 and 222 around the rotation shaft 230, β is the angle obtained by subtracting the angle formed by the first lever arm 221 and an imaginary line perpendicular to the ground from 90 degrees, θ is the angle formed by the horizontal line and the second lever arm 222, τ is the magnitude of the torque measured by the sensor unit 240, L is the length from the rotation shaft 230 to the end of the second contact unit 212, m is the weight of the upper or lower limb, and g is the acceleration of gravity. Since the magnitude τ of the torque measured by the sensor unit 240 is mgLcosθ, the weight of the upper or lower limb in the state in which no muscle force is exerted is calculated as m=τ/gLcosθ.

The wearable muscle strength measuring device performs a gravity compensation mechanism on the wearable muscle strength measuring device by adjusting the zero point of the sensor unit 240 based on the measured weight in step S120.

The actual muscle strength may be 0, and the sensing value may be 0. In this case, the wearable muscle strength measuring device measures the weight. In the case where the muscle strength is not 0, in the isometric muscle strength measurement mode, in order to output the same sensing value, a subject having a higher upper or lower limb weight needs to exert a higher force than a subject having a lower upper or lower limb weight. In other words, the sensing values are the same, but different muscle strengths are exerted. Accordingly, as for the sensing value of the subject having a higher upper or lower limb weight, the gravity compensation mechanism is performed such that a higher muscle strength is calculated compared to the sensing value with a weight value corresponding to the weight of the upper or lower limb reflected therein.

The wearable muscle strength measuring device measures the muscle strength of the upper or lower limb that resists the movement of the lever arms that rotate around the rotation shaft 230 of the wearable muscle strength measuring device, measures the muscle strength of the upper or lower limb at a preset angle corresponding to the type of joint of the subject in step S130.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A wearable muscle strength measuring device comprising:
a rotation shaft configured to be located on a joint part of a subject;
a first lever arm extending in a first direction based on the rotation shaft;
a second lever arm extending in a second direction different from the first direction based on the rotation shaft;
a first contact part fixed to the first lever arm and formed into a curved surface so that a proximal portion of an upper or lower limb of a subject is seated thereon;
a second contact part fixed to the second lever arm and formed into a curved surface so that a distal portion of the upper or lower limb of the subject is seated thereon, and having an area smaller than or equal to that of the first contact part; and
sensor unit coupled to the rotation shaft or the second lever arm, and configured to measure at least one of a muscle strength of the upper or lower limb and a weight of the upper or lower limb.

2. The wearable muscle strength measuring device of claim 1, further comprising:
a control unit configured to perform a gravity compensation mechanism by adjusting a zero point of the sensor unit based on the weight of the upper or lower limb measured in a state where a muscle strength of the subject is not exerted and calculate the muscle strength of the upper or lower limb.

3. The wearable muscle strength measuring device of claim 2, further comprising:
a motor unit configured to spread apart the first and second lever arms while rotating them at a preset angular velocity around the rotation shaft;
wherein the sensor unit measures the muscle strength of the upper or lower limb that resists movement of the first and second lever arms.

4. The wearable muscle strength measuring device of claim 2, wherein the sensor unit measures the muscle strength of the upper or lower limb at a preset angle corresponding to a type of joint of the subject.

5. The wearable muscle strength measuring device of claim 4, further comprising:
a ratchet gear having the rotation shaft as its central axis; and
a stopper configured to perform hooking engagement with the ratchet gear at the preset angle.

6. The wearable muscle strength measuring device of claim 4, wherein the sensor unit comprises a load cell including a strain gauge, a fixation portion of the load cell is located on the rotation shaft, and a force application point of the load cell is located on a bottom surface of the second contact part.

7. The wearable muscle strength measuring device of claim 2, wherein the first and second contact parts each further comprise:
a cylindrical frame configured such that the upper or lower limb of the subject is inserted thereinto; and
a close contact part provided inside the frame so that the upper or lower limb of the subject is maintained in contact while the upper or lower limb of the subject moves.

8. The wearable muscle strength measuring device of claim 7, further comprising:
a plurality of close contact sensors spaced apart at preset intervals inside the close contact part to detect whether the upper or lower limb is maintained in contact.

9. The wearable muscle strength measuring device of claim 8, wherein the close contact part comprises a cushion including a non-Newtonian liquid or an air pressure pump, and the cushion is deformed in accordance with a thickness of the upper or lower limb of the subject.

10. The wearable muscle strength measuring device of claim 2, further comprising:
a support configured to fix the wearable muscle strength measuring device so that a direction of movement of the joint of the subject is perpendicular to a direction of gravity;
wherein the control unit modifies the gravity compensation mechanism according to a location at which the support is installed and a form in which the wearable muscle strength measuring device is coupled with the support.

11. The wearable muscle strength measuring device of claim 2, further comprising:
a display unit configured to display a magnitude of at least one of the measured value, measured by the sensor unit, and the muscle strength of the upper or lower limb, calculated by the control unit, according to movement of the subject, as a color code and distinctively display a point at which the magnitude is maximum.

12. The wearable muscle strength measuring device of claim 2, wherein each of the first and second contact parts comprises:
a plurality of support plates each formed as a curved surface, and each provided with slots through which straps for fixing the upper or lower limb pass; and
length adjustment parts configured to connect the individual support plates.

13. The wearable muscle strength measuring device of claim 1, wherein the first and second lever arms are folded to face a same direction based on the rotation shaft.

14. A muscle strength measuring method, the muscle strength measuring method being performed by a wearable muscle strength measuring device, the muscle strength measuring method comprising:
measuring a weight of an upper or lower limb of a subject in a state where the subject's muscle strength is not exerted;
performing a gravity compensation mechanism on the wearable muscle strength measuring device by adjusting a zero point of a sensor unit based on the measured weight; and
measuring the muscle strength of the upper or lower limb that resists movement of lever arms rotating around the rotation shaft of the wearable muscle strength measuring device, or measuring the muscle strength of the upper or lower limb at a preset angle corresponding to a type of joint of the subject.

15. The wearable muscle strength measuring method of claim 14, wherein measuring the weight of the upper or lower limb comprises:
checking whether a direction of movement of the joint of the subject and a direction of gravity are perpendicular to each other; and
when they are perpendicular, determining a value measured by the sensor unit to be the weight of the upper or lower limb, and, when they are not perpendicular, measuring an angle formed by the direction of movement of the joint of the subject and the direction of gravity, and calculating the weight of the upper or lower limb based on the angle and the value measured by the sensor unit.
